(19) United States
(12) Patent Application Publication
(10) Pub. No.: US 2020/0323676 A1
(43) Pub. Date: Oct. 15, 2020

(54) LEG SLING

(71) Applicant: LE Sling LLC, Moore, OK (US)

(72) Inventor: Amy LEE, Moore, OK (US)

(21) Appl. No.: 16/382,745

(22) Filed: Apr. 12, 2019

(51) Int. Cl. A61F 5/37

(52) U.S. Cl. CPC A61F 5/3715

(57) ABSTRACT

A leg sling for use on a lower limb has a waist belt configured to be secured around a user's waist. At least two connectors are attached to the waist belt. A pair of assistance lines made of a stretchable and resilient material are provided. Each of the assistance lines has a connector end attachable to one of the at least two connectors and a foot sling end. A foot sling is made of a flexible material. The foot sling has a foot support, a left connection and a right connection. The left connection is connectable to the foot sling end of one of the assistance lines and the right connection is connectable to the foot sling end of the other assistance line.

### FIELD OF THE DISCLOSURE

The present application relates generally to a leg sling, more particularly it relates to a leg sling for assisting through push off and swing phases of a gait cycle.

### BACKGROUND

This section provides background information to facilitate a better understanding of the various aspects of the invention. It should be understood that the statements in this section of this document are to be read in this light, and not as admissions of prior art.

When a leg is injured, it is common for a person to seek support to assist with the healing process. Crutches and scooters are commonly used to assist those with leg injuries with mobility, however finding assistance when the limb is ready to bear weight again can be a challenge. With limited or non-use, muscles in a leg will weaken while the limb heals. When it is time to begin using the limb again, people can struggle with creating a proper leg swing while also maintaining proper clearance of the foot during the swing. This can result in additional injuries, reinjuries or additional rehab time. While different devices have been created to assist with these issues, they can be costly, have antiquated designs and be difficult to position on a user without assistance. Many of them are also incapable of being properly adjusted as the user requires varying degrees of assistance over time.

### BRIEF SUMMARY

There is provided a leg sling for use on a lower limb. A waist belt is configured to be secured around a user's waist and has at least two connectors that attach to the waist belt. A pair of assistance lines made of a stretchable and resilient material are provided. Each of the assistance lines has a connector end attachable to one of the at least two connectors and a foot sling end. A foot sling is made of a flexible material and has a foot support, a left connection and a right connection. The left connection is connectable to the foot sling end of one of the assistance lines and the right connection portion is connectable to the foot sling end of the other assistance line.

In one embodiment, there are two connectors attached to the waist belt. In a preferred embodiment, the two connectors are removably attached to the waist belt. This allows the connectors to be positioned based upon the needs of the user and allows the leg sling to be used by people of different sizes.

In one embodiment, one of the connectors is positioned at a central point at a front of the waist belt and the other connector is positioned on a side of the belt corresponding to a leg of the user being supported by the foot sling. This orientation of connectors places assistance lines in a suitable position to assist with the swing phase of the gait and assist in providing proper clearance of the foot during the swing.

In one embodiment, the at least two connectors each have a belt connecting assembly and a tension device. The belt connecting assembly has a belt connection end and a tension device end. The tension device is engageable with the tension device end of the belt connecting assembly. The tension device has a tensioner for attaching to the connector end of one of the assistance lines and allowing for tensioning of one of the assistance lines based upon a user's preference.

In one embodiment, the belt connection end of the belt connecting assembly is a set of gripping jaws. The gripping jaws may be tightened or loosened through the use of a tightener. The tightener may be a screw lock or any other suitable tightener known to a person skilled in the art. The gripping jaws may have teeth that can enhance the ability of the belt connection end to grip the waist belt.

In one embodiment, the tension device has a body with a carabiner hook and a closing member that is pivotable between an open position and a closed position. The carabiner hook is connectable to the tension device end of the belt connecting assembly. A cord gripper is attached to the body. The cord gripper has an aperture through which the connector end of one of the assistance lines is inserted. The cord gripper has a first channel of a first side of the cord gripper and a second channel on the second side of the cord gripper into which the assistance line is placed. A tensioning wheel is positioned such that it extends a portion of the way into the second channel causing a narrowing of the second channel. The tensioning wheel rotates to pinch the assistance line between the tension wheel and a side of the second channel.

In one embodiment, the tensioning wheel has teeth to grip the assistance line. A finger tab may also be provided on the tensioning wheel to assist the user in rotating the tensioning wheel.

In one embodiment, the pair of assistance lines are bungees.

In one embodiment, the tension device is made of metal. The metal may prevent bending, stretching or misshaping of the tensioning device due to the pressures exerted on it during use.

In one embodiment, the diameter of the waist belt is adjustable. The waist belt may be made of nylon or any other suitable material.

In one embodiment, the foot support is textured to create a grip between the foot support and the user's foot, sock or shoe.

In one embodiment, the foot sling has an upper strap that extends from the right connection to the left connection. Upper strap helps to prevent a user's foot from moving out of foot sling during movement of the leg.

### BRIEF DESCRIPTION OF THE DRAWINGS

These and other features will become more apparent from the following description in which references are made to the following drawings, in which numerical references denote like parts. The drawings are for the purpose of illustration only and are not intended to in any way limit the scope of the invention to the particular embodiments shown.

FIG. 1 is a side elevation view of a leg sling positioned on a user's right leg.

FIG. 2 is a front elevation view of the leg sling positioned on a user's right leg.

FIG. 3 is a perspective view of the leg sling.

FIG. 4 is a perspective view, partially in section, of an embodiment of a foot sling.

FIG. 5 is a perspective view of a waist belt with connectors.

FIG. 6 is a bottom plan view of an embodiment of the foot sling.

FIG. 7 is a top plan view of the embodiment of the foot sling shown in FIG. 6.

FIG. 8 is a bottom plan view of the embodiment of the foot sling shown in FIG. 4.

FIG. 9 is a top plan view of the embodiment of the foot sling shown in FIG. 4.

FIG. 10 is a first side elevation view of a tensioning device.

FIG. 11 is a second side elevation view of the tensioning device.

### DETAILED DESCRIPTION OF THE PREFERRED EMBODIMENTS

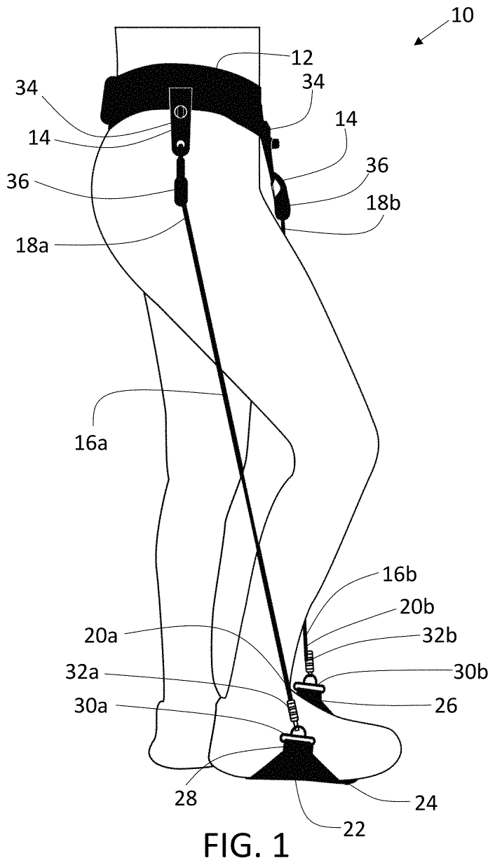

A leg sling, generally identified by reference numeral 10, will now be described with reference to FIG. 1 through FIG. 11.

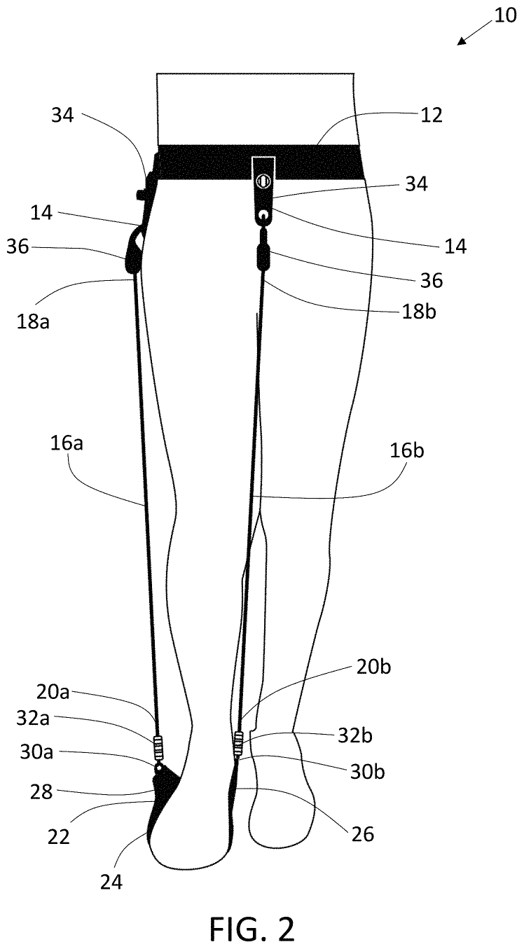

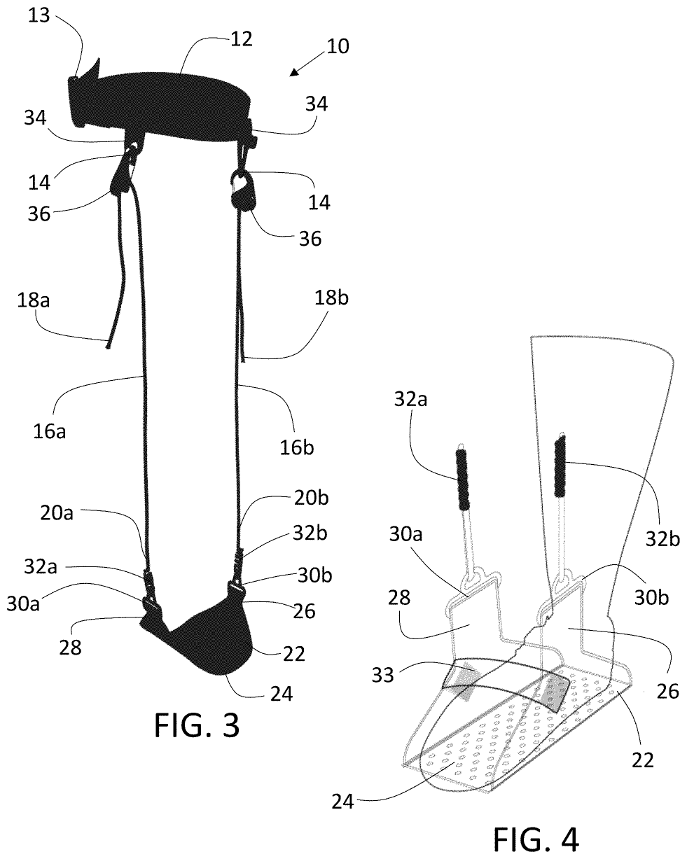

Referring to FIG. 1, a leg sling 10 has a waist belt 12 configured to be secured around a user's waist. Waist belt 12 is preferably adjustable in size and made of nylon. In the embodiment shown, waist belt 12 is made of a 2 inch wide black belt webbing made of a blend of nylon and polypropylene with a thickness of approximately 0.114 inches. It will be understood by a person skilled in the art that waist belt 12 may be made of other suitable materials or dimensions. Referring to FIG. 3, in the embodiment shown, waist belt 12 has a releasable buckle 13 to allow for ease when placing belt on and removing belt from a user's waist. It will be understood that different types of buckles and connections may be used for securing waist belt 12. Connectors 14 are attached to waist belt 12. In the embodiment shown, there are two connectors 14 that are removable to allow connectors 14 to be positioned based upon the user's preference. It will be understood that connectors 14 may be fixedly attached to waist belt 12 and that a plurality of connectors 14 may be used to allow the user to use pre-positioned connectors. In the embodiment shown in FIG. 2, one connector 14 is positioned at a central point at a front of waist belt 12 and a second connector 14 is positioned on a side of waist belt 12 corresponding to a leg of the user being supported. Releasable buckle 13, not shown in FIG. 2, is positioned at a rear of the user so as to not interfere with positioning of connectors 14.

Referring to FIG. 3, a pair of assistance lines 16a and 16b are provided. Each of assistance lines 16a and 16b is made of a stretchable and resilient material. In the embodiment shown, assistance lines 16a and 16b are made of bungees, however it will be understood by a person skilled in the art that different types of assistance lines 16a and 16b may be used. In one embodiment, assistance lines 16a and 16b are 3/16 inch diamond weave bungees with a UV, fray, rot, mold and mildew resistant cover. Tensile strength of this type of bungee is 190 pounds. It will be understood by a person skilled in the art that different types of bungees may be used and different tensile strengths may be required for larger or smaller users. Each of assistance lines 16a and 16b have a connector end 18a and 18b attachable to a connector and a foot sling end 20a and 20b.

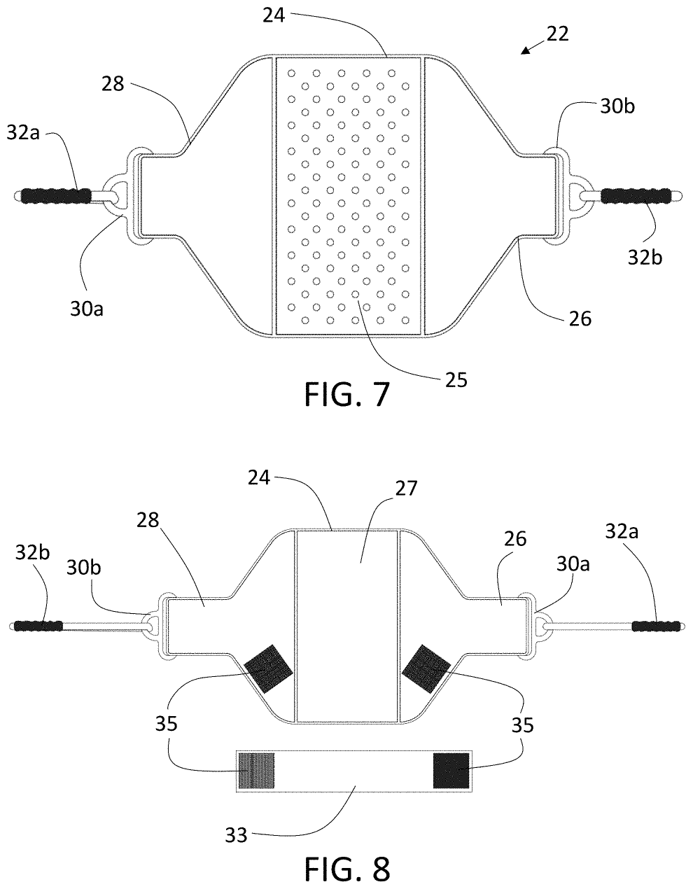

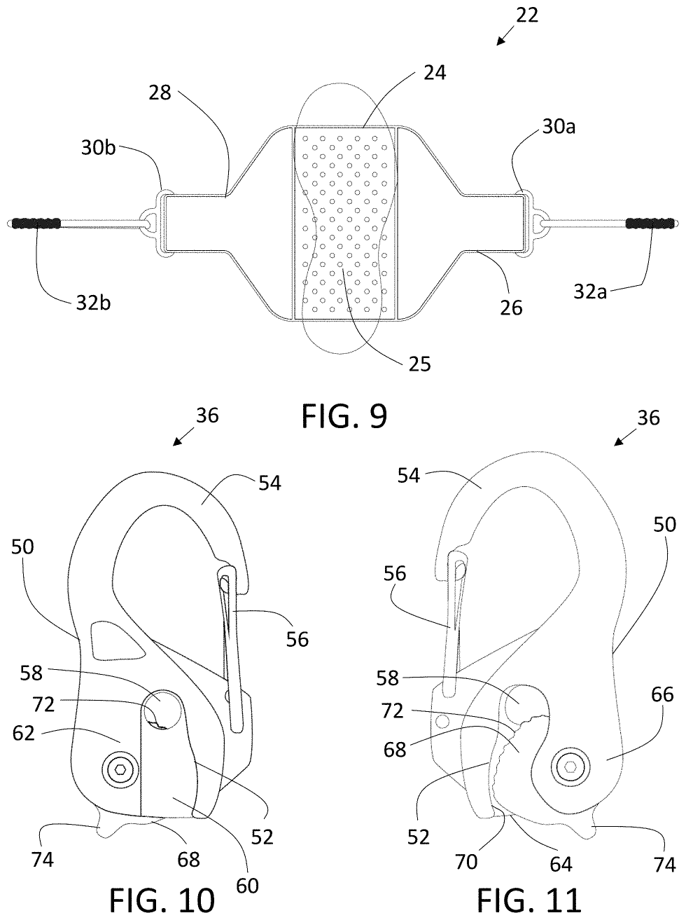

A foot sling 22 is made of a flexible material such as moisture-repellant leatherette padded fabric. It will be understood that other types of materials may be used. Foot sling 22 may be worn inside or outside of shoes. Foot sling 22 has a foot support 24, a left connection 26 and a right connection 28. Foot support 24 may have a textured surface to provide gripping for the foot, shoe or sock in contact with it. As can be seen in FIG. 7 and FIG. 9, upper surface 25 is textured while lower surface 27, shown in FIG. 6, is not textured. It will be understood that both upper surface 25 and lower surface 27 may be textured or that neither upper surface 25 nor lower surface 27 may be textured. Left connection 26 is connectable to foot sling end 20a of assistance line 16a and right connection 28 is connectable to foot sling end 20b of assistance line 16b. In the embodiment shown, assistance lines 16a and 16b are permanently attached to left connection 26 and right connection 28 through the use of d-rings 30a and 30b and hand-crimped copper swages 32a and 32b. It will be understood by a person skilled in the art that assistance lines 16a and 16b may be removably attached to foot sling 22 or attached in any other manner known to a person skilled in the art. As can be seen in FIG. 6-9, the size and shape of left connection 26 and right connection 28 may vary. Referring to FIG. 4 and FIG. 8, an upper strap 33 may extend from right connection 28 to left connection 26 for helping to prevent a user's foot from moving out of foot sling during movement of the leg. Upper strap 33 may be fixedly attached to right connection 28 and left connection 26, however for convenience of use it is preferred that upper strap 33 be removably attached. In the embodiment shown, Velcro strips 35 are used for removably attaching upper strap 33 to right connection 28 and left connection 26. It will be understood by a person skilled in that art that buckles and other methods of attaching upper strap 33 to right connection 28 and left connection 26 may be used. One side of upper strap 33 may be fixedly attached to one of right connection 28 and left connection 26 while the other side of upper strap is removably attached to the other of right connection 28 and left connection 26.

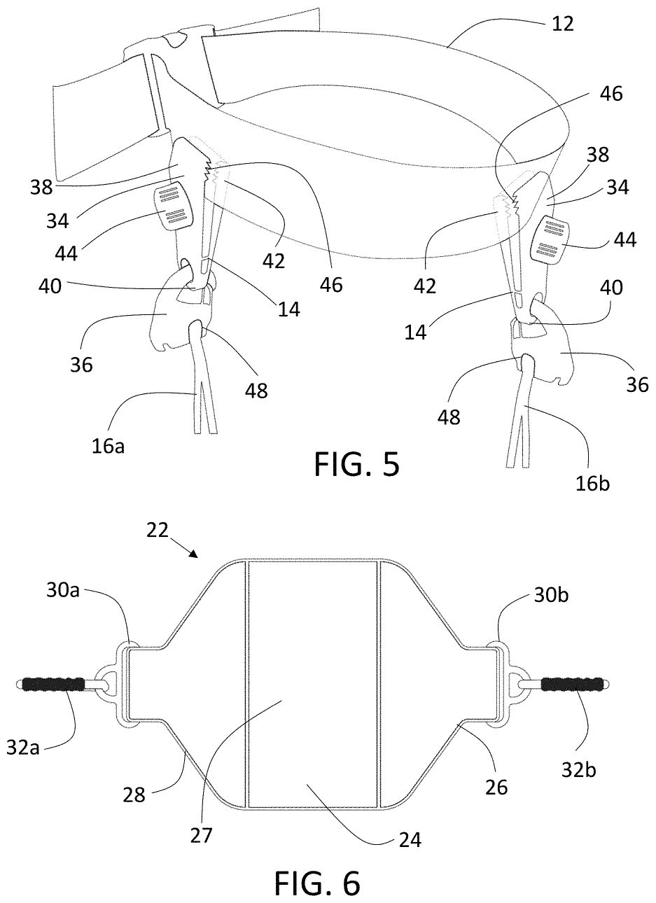

Referring to FIG. 5, in the embodiment shown, connectors 14 each have a belt connecting assembly 34 and a tension device 36. Belt connecting assembly 34 has a belt connection end 38 and a tension device end 40. In the embodiment shown, belt connection end 38 of belt connecting assembly 34 is a set of gripping jaws 42 which may be tightened or loosened through the use of a tightener 44 such as a screw lock or other tightener known to a person skilled in the art. Gripping jaws 42 may have teeth 46 that allow for improved gripping to waist belt 12 as teeth 46 bite into waist belt 12. Tension device 36 is engageable with tension device end 40 of belt connecting assembly 34. Tension device 36 has a tensioner 48 for attaching to connector end 18a or 18b of assistance line 16a or 16b and allowing for tensioning based upon a user's preference.

Referring to FIG. 10 and FIG. 11, tension device 36 has a body 50 and a cord gripper 52. Body 50 has a carabiner hook 54 with a closing member 56 that is pivotable between an open position and a closed position. Carabiner hook 54 is connectable to tension device end 40 of belt connecting assembly 34. Cord gripper 52 is attached to body 50. Cord gripper 52 has an aperture 58 through which connector end 18a and 18b of assistance lines 16a and 16b is inserted. Referring to FIG. 10, cord gripper 52 has a first channel 60 on a first side 62 of cord gripper 52. Referring to FIG. 11, cord gripper 52 has a second channel 64 on a second side 66 of cord gripper 52. Referring to FIG. 5, assistance lines 16a and 16b are placed within first channel 60 and second channel 64, shown in FIG. 10 and FIG. 11, respectively. Referring to FIG. 11, a tensioning wheel 68 is positioned such that it extends a portion of the way into second channel 64 causing a narrowing of second channel 64. Tensioning wheel 68 is rotatable to pinch assistance line 16a or 16b between tensioning wheel 68 and a side 70 of second channel 64. Tensioning wheel 68 may have teeth 72 to grip assistance line 16a or 16b. A finger tab 74 is positioned on tensioning wheel 68 to aid in rotating tensioning wheel 68. Tension device 36 may be made of metal. The metal may prevent bending, stretching or misshaping of the tensioning device due to the pressures exerted on it during use. It will be understood by a person skilled in the art that tension device 36 may be made of any suitable material.

Referring to FIG. 2, as can be seen in the embodiment shown, assistance lines 16a and 16b do not cross each other. When used for supporting a right leg, connectors 14 are positioned on waist belt 12 at the right side of the waist and at a central position on the front of waist belt 12. Assistance line 16a connects to connector 14 positioned at the right side of the waist and right connection 28 of foot sling 22. Assistance line 16b connects to connector 14 positioned at a central position on the front of waist belt 12 and left connection 26 of foot sling 22. Connectors 14 may be moved to alternate locations depending upon the challenges faced by each user. A user may find it more comfortable to move connectors 14 closer to each other, farther apart, off center, towards the rear of waist belt 12 or any other position suited to the user.

Any use herein of any terms describing an interaction between elements is not meant to limit the interaction to direct interaction between the subject elements, and may also include indirect interaction between the elements such as through secondary or intermediary structure unless specifically stated otherwise.

In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be one and only one of the elements.

It will be apparent that changes may be made to the illustrative embodiments, while falling within the scope of the invention. As such, the scope of the following claims should not be limited by the preferred embodiments set forth in the examples and drawings described above, but should be given the broadest interpretation consistent with the description as a whole.

What is claimed is:

1. A leg sling comprising:
a waist belt configured to be secured around a user's waist;
at least two connectors attached to the waist belt;
a pair of assistance lines, each of the assistance lines being made of a stretchable and resilient material, each of the assistance lines having a connector end attachable to one of the at least two connectors and a foot sling end;
a foot sling being made of a flexible material, the foot sling having a foot support, a left connection and a right connection, the left connection being connectable to the foot sling end of one of the assistance lines and the right connection being connectable to the foot sling end of the other assistance line.

2. The leg sling of claim 1 wherein there are two connectors attached to the waist belt.

3. The leg sling of claim 2 wherein the two connectors are removably attached to the waist belt.

4. The leg sling of claim 1 wherein one of the connectors is positioned at a central point at a front of the waist belt and the other connector is positioned on a side of the belt corresponding to a leg of the user being supported by the foot sling.

5. The leg sling of claim 1 wherein the at least two connectors each comprise:
a belt connecting assembly having a belt connection end and a tension device end;
a tension device engageable with the tension device end, the tension device having a tensioner for attaching to the connector end of one of the assistance lines and allowing for tensioning of one of the assistance lines based upon a user's preference.

6. The leg sling of claim 5 wherein the belt connection end of the belt connecting assembly is a set of gripping jaws, the gripping jaw being tightened or loosened through a tightener.

7. The leg sling of claim 6 wherein the tightener is a screw lock.

8. The leg sling of claim 6 wherein the gripping jaws have teeth for gripping the waist belt.

9. The leg sling of claim 5 wherein the tension device comprises:
a body having a carabiner hook with a closing member being pivotable between an open position and a closed position, the carabiner hook being connectable to the tension device end of the belt connecting assembly;
a cord gripper attached to the body, the cord gripper having an aperture through which the connector end of one of the assistance lines is inserted, the cord gripper having a first channel on a first side of the cord gripper and a second channel on a second side of the cord gripper into which the assistance line is placed, a tensioning wheel is positioned such that it extends a portion of the way into the second channel causing a narrowing of the second channel, the tensioning wheel being rotatable to pinch the assistance line between the tensioning wheel and a side of the second channel.

10. The leg sling of claim 9 wherein the tensioning wheel has teeth to grip the assistance line.

11. The leg sling of claim 9 wherein the tensioning wheel has a finger tab to rotate the tensioning wheel

12. The leg sling of claim 1 wherein the pair of assistance lines are bungees.

13. The leg sling of claim 5 wherein the tension device is made of metal.

14. The leg sling of claim 1 wherein the diameter of the waist belt is adjustable.

15. The leg sling of claim 1 wherein the waist belt is made of nylon.

16. The leg sling of claim 1 wherein the foot support is textured.

17. The leg sling of claim 1 wherein the foot sling further comprises an upper strap extending from the right connection to the left connection.
